(19) United States
(12) Patent Application Publication
(10) Pub. No.: US 2021/0270828 A1
(43) Pub. Date: Sep. 2, 2021

(54) METHOD FOR SEEDING CELLS ON A SENSOR SURFACE

(71) Applicants: Hoffmann-La Roche Inc., Little Falls, NJ (US); NCL New Concept Lab GmbH, Moehlin (CH)

(72) Inventors: Stephen FOWLER, Basel (CH); Na Hong QIU, Basel (CH); Guojun CHEN, Moehlin (CH)

(73) Assignees: Hoffmann-La Roche Inc., Little Falls, NJ (US); NCL New Concept Lab GmbH, Moehlin (CH)

(21) Appl. No.: 17/324,985

(22) Filed: May 19, 2021

Related U.S. Application Data
(63) Continuation of application No. PCT/EP2019/081579, filed on Nov. 18, 2019.

(30) Foreign Application Priority Data
Nov. 20, 2018 (EP) 18207179.5

(51) Int. Cl. G01N 33/543; G01N 33/50

(52) U.S. Cl. CPC G01N 33/54366; G01N 33/5008

(57) ABSTRACT

The present invention provides a method for attaching cells to a biosensor surface (5) of a biosensor (4) comprising: providing a cell suspension in a liquid receiving unit (1), wherein the cell suspension forms a surface (7) to the exterior of the liquid receiving unit (1); contacting the biosensor surface (5) with the surface (7) of the cell suspension in the liquid receiving unit (1); and allowing the cells to settle on the biosensor surface (5) by gravity, and allowing the cells to adhere to the biosensor surface (5).

### CROSS-REFERENCE TO RELATED APPLICATIONS

This application is a continuation of International Application No. PCT/EP2019/081579, filed Nov. 18, 2019, which claims priority to EP Application No. 18207179.5, filed Nov. 20, 2018, which are incorporated herein by reference in their entireties.

The present invention relates to a method for seeding cells on a biosensor surface and the use of the seeded cells in methods to measure molecule cell interactions.

Associating living cells with biosensors is technically challenging, due to the tiny area of the biosensor tip. When placing the biosensor face-up, the very small tip area cannot hold enough cell culture medium and will dry out very rapidly. It is also impossible to place the biosensor face-down as cell sedimentation causing the cells to drop to the bottom of the media, moving away from the biosensor to which they should become attached. Current approaches involve changing the density of the culture medium to prevent cells from sinking with time potentially allowing them to interact with the sensor surface and bind [reference paper]. The reagents for achieving this are still under the development and are not commercially available due to the quality issues such as cell culture condition changes, cell toxicity of the reagents, and inconsistent results of cell attachment.

Therefore, a simple and robust method is needed to allow living cells to associate with the biosensors in the optimal cell culture condition.

In a first aspect, the present invention provides a method for attaching cells to a biosensor surface (5) of a sensor (4) comprising:
a) providing a cell suspension in a liquid receiving unit (1), wherein the cell suspension forms a surface (7) to the exterior of the liquid receiving unit (1),
b) contacting the biosensor surface (5) with the surface (7) of the cell suspension in the liquid receiving unit (1) and
c) allowing the cells to settle on the sensor surface (5) by gravity, and allowing the cells to adhere to the biosensor surface (5).

In an embodiment of the method of the present invention, the liquid receiving unit (1) keeps the cell suspension in a defined area/space through adhesion force and surface tension.

In an embodiment of the method of the present invention, the liquid receiving unit (1) comprises a structure selected from the group consisting of a capillary tube, a micro-groove, a micro-well, a micro-loop, a micro-wire spring, or a micro-protrude.

In an embodiment of the method of the present invention, the liquid receiving unit (1) comprises a capillary tube (3) which is connected to a reservoir (2) to form a liquid receiving unit (1).

In an embodiment of the method of the present invention, the capillary tube (3) has an end opening (6) at which the cell suspension forms the surface (7) to the exterior of the liquid receiving unit (1) and said surface (7) is in contact with the sensor surface (5).

In an embodiment of the method of the present invention, the capillary tube (3) has a hydrophobic zone at the end opening (6) to prevent the cell suspension from draining off.

In an embodiment of the method of the present invention, the capillary tube (3) is at least filled with the cell suspension.

In an embodiment of the method of the present invention, the liquid receiving unit (1) is arranged in array format comprising more than one liquid receiving unit (1), preferably the array format is a 96 unit plate format, more preferably a 96 unit IMAPlate™.

In an embodiment of the method of the present invention, the sensor (4) is a needle-like sensor with a sensor surface (5).

In an embodiment of the method of the present invention, the biosensor surface (5) is placed face-up.

In an embodiment of the method of the present invention, in step c) the cells are allowed to settle for about 1-24 hours.

In an embodiment of the method of the present invention, the biosensor surface (5) is coated with a biocompatible matrix to support cell attachment and cell growth.

In an embodiment of the method of the present invention, the biosensor surface (5) is pretreated with solvent such as aceton before coated with the biocompatible matrix.

In an embodiment of the method of the present invention, the biosensor surface (5) is coated with molecules which specifically interact with surface molecules of the cells to be immobilized on the sensor surface (5).

In a second aspect, the present invention provides a method for measuring molecular interactions between a test molecule and cells comprising:
a) immobilizing cells on a biosensor surface (5) according to the method for attaching cells to a biosensor surface of a sensor according to the present invention,
b) incubating the sensor (4) with the test molecule and
c) measuring interaction of the test molecule with the immobilized cells by appropriate methods.

In an embodiment of the method of the present invention, the molecule is a bio-molecule.

In an embodiment of the method of the present invention, the appropriate method is Bio-layer interferometry.

In an embodiment of the method of the present invention, the test molecules are in reaction chambers of a multi well plate, preferably a 96 multi well plate.

In a third aspect, the present invention provides a kit for attaching cells to a biosensor surface (5) of a sensor (4) comprising a multi-unit plate comprising a plurality of liquid receiving units (1), wherein the liquid receiving units (1) have a reservoir part (2) and a capillary part (3) with an end opening (6), a set of biosensors (4) and a protocol for a method to attach cells to the biosensor surface (5) according to the cell seeding method of the present invention.

In an embodiment of the kit of the present invention, the multi-unit plate is a IMAPlate™ and the sensor is a Bio-layer interferometry biosensor.

In an embodiment of the present invention, the kit further comprises a multi-unit plate which accommodates the set of biosensors (4) and spacers to connect the two multi-unit plates.

IMAPlate™ is a registered trademark from NCL New Concept Lab GmbH.

IMAPlate™ are commercially available from different sources such as e.g. NCL New Concept Lab GmbH, CH-4313 Moehlin.

The term “test compound” as used herein comprises organic or inorganic compounds, derived synthetically or from natural sources. The compounds include inorganic or organic compounds such as, but not limited to, polynucleotides, lipids, polysaccharide or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows an exemplary assembly to perform the cell seeding method of the present invention. The assembly comprises an IMAplate™ with a plurality of liquid receiving units 1 and a biosensor holder which accommodates the biosensors 4 comprising a sensor surface 5 to be seeded with cells. The two plates are hold in a defined distance by four spacers arranged at the four corners of both plates. The liquid receiving units 1 of the upper plate comprise a lower capillary tube part 3 and an upper reservoir part 2. The lower capillary part has an open ending with a hydrophobic zone to prevent the cell suspension from draining off. The defined distance between the two plates brings the sensor surface 5 in contact with the cell suspension surface 7 formed at the lower end 6 of capillary tube 3.

FIG. 2 shows a sensor surface 5 with cells seeded according to the method of the present invention. The cells form a monolayer on the sensor surface.

FIG. 3A-FIG. 3E show different embodiments of liquid receiving units 1 according the present invention. FIG. 3A shows a liquid receiving unit 1 comprising an upper reservoir part 2 and a lower capillary tube part 3. The capillary tube part 3 has an open bottom 6 and the opening zone of the capillary tube 3 is made of hydrophobic material such as e.g. polystyrol, to prevent the liquid containing the cells to be seeded from draining off.

FIG. 3B-FIG. 3E show additional embodiments of liquid receiving units of the invention:

FIG. 3B: a micro-groove, functioning as a capillary with an open wall; FIG. 3C: a micro-loop; FIG. 3D: a micro-wire spring and FIG. 3E: a micro-protrude.

FIG. 4 shows a sensor 4—liquid receiving unit 1 assembly according to an embodiment of the present invention. The liquid receiving unit 1 comprises a reservoir part 2 and a capillary tube 3 with an end opening 6. The liquid receiving unit 1 is depicted in its filled state i.e. the liquid receiving unit 1 is filled with a cell suspension. The cell suspension in the liquid receiving unit 1 forms at the end opening 6 of the capillary tube 3 a surface to the exterior 7, in particular a convex meniscus 7, which is brought in contact with the sensor surface 5.

FIG. 5 depicts a magnified view of the interface between the convex liquid meniscus 7 formed at the end opening 6 of the capillary tube 3 and the sensor surface 5 of the sensor/liquid receiving unit assembly shown in FIG. 4.

FIG. 6 shows the results of an antibody binding kinetic experiment using a biosensor coated with cells according the method of the present invention in Bio-layer interferometry (BLI).

The method of the present invention allows efficient cell seeding onto a needle-like biosensor surface using normal cell culture media. The inventive method allows a fine control of cell seeding density and no special reagents are required preventing cell stress.

The inventive method can be used with any needle-like sensor system where cells or particles need to be immobilized on a biosensor surface. The immobilized cells or particles can be used for biophysical measurements of small and large molecules and oligonucleotide compound interactions with cells or particles. Suitable biosensors are commercially available from FORTEBIO (www.fortebio.com).

### EXAMPLES

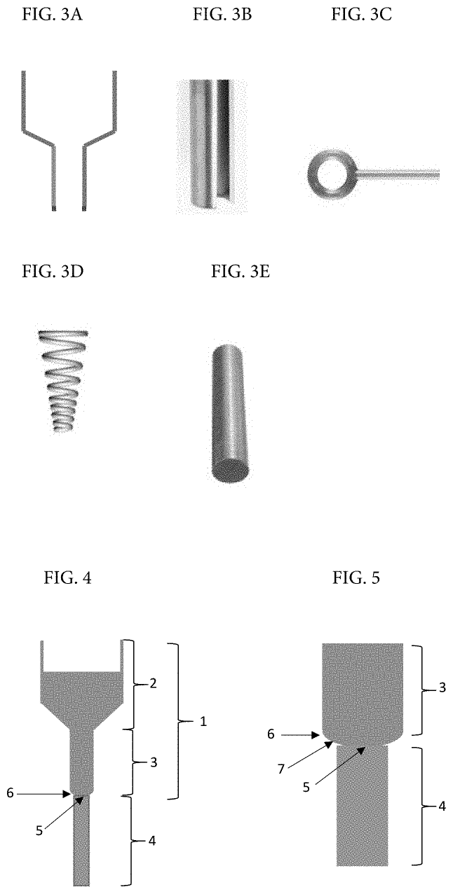

### Example 1: Seeding of Cells on a Biosensor Surface Using an IMAPlate™ with 96 Liquid Receiving Units Having the Configuration Depicted in FIG. 3A and FIG. 4. The Seeding Method Comprises the Following Steps

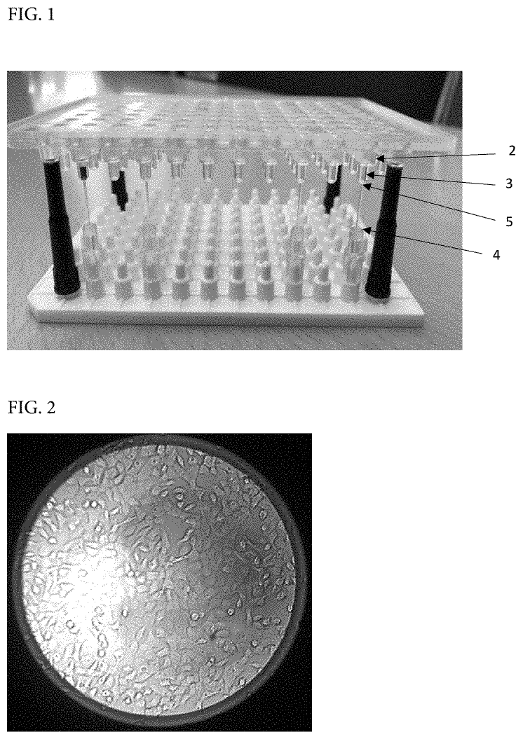

1. Preparing a cell suspension using a standard protocol of passaging adherent cells.
2. Diluting the cell suspension with cell culture medium to a proper cell density (typical range: 0.5×10⁶ to 1×10⁶ cells/mL or 2000 to 5000 cells per biosensor surface).
3. Assemble the cell seeding assembly as shown in FIG. 1. The assembly comprises an upper IMAPlate with 96 liquid receiving units 1 filled with the cell suspension and a lower plate accommodating the needle sensors 4 with sensor surface 5.
4. Mixing the cell suspension and loading 5 μL of the cell suspension to the capillary tube 3 of the upper IMAPlate, then cover the 5 μl cell suspension in the capillary tube 3 by loading 20 to 30 μL cell culture medium in the reservoir part 2 of the liquid receiving unit 1. The cell suspension in the capillary tube 3 forms a surface at the end opening of the capillary tube to the exterior but does not drain off. The sensor surface 5 is brought in contact with the liquid surface and the cells can seed and adhere to the sensor surface 5.
5. Incubation for 2 to 4 hours in a cell culture incubator to allow the cells to settle down and adhere to the sensor surface 5.
6. Remove the sensor 4 from the cell seeding assembly; place the sensor surface 5 with adhered cells (face down) into a well of a standard 96 well plate containing cell culture medium and incubate overnight in a cell culture incubator.
7. Check biosensor surface for cell morphology. The biosensor with adhered cells on its surface 5 in now ready for use in cell—molecules binding assays.

### Example 2: Coating of a Biosensor Surface with a Biocompatible Matrix (Collagen)

The inventors found that commercially available sensors directly coated with biocompatible matrix could hardly support cell attachment and grow. It was probably due to the toxicity of the material on the biosensor surface. After many trials, we found that pretreating with solvent such as acetone before biocompatible matrix coating can allow cell to grow normally.

In an embodiment of the invention, the biosensor surface is coated with a biocompatible matrix to improve cell adhesion to the biosensor surface. An exemplary method to coat the biosensor surface with the biocompatible matrix comprises the following steps:
1. Place the biosensors (surface down) in wells or tubes containing acetone, make sure that the biosensor surface is in contact with acetone.
2. Incubation of the biosensors at RT (20° C.) for about 15 mins with slightly stirring.
3. Transfer the biosensors into new wells or tubes containing acetone and incubate as in step 2.
4. Repeat the step 3.
5. Transfer the acetone treated biosensors in wells or tubes containing ethanol, incubate about 5 mins with slightly stirring.
6. Finally wash the biosensors with water. Now the biosensors are ready to be coated with collagen.

Coating of Biosensor Surface with Collagen:
a) Place the biosensors (surface down) in wells or tubes containing collagen solution (typically at 0.1-0.5 mg/mL concentration), make sure the biosensor surface is in contact with the liquid.
b) Incubate overnight at RT (20° C.), and dry the biosensors overnight at RT (20° C.).
c) Wash the biosensors with PBS, followed by water. Now the biosensors are ready to be seeded with cells by the method of the present invention.

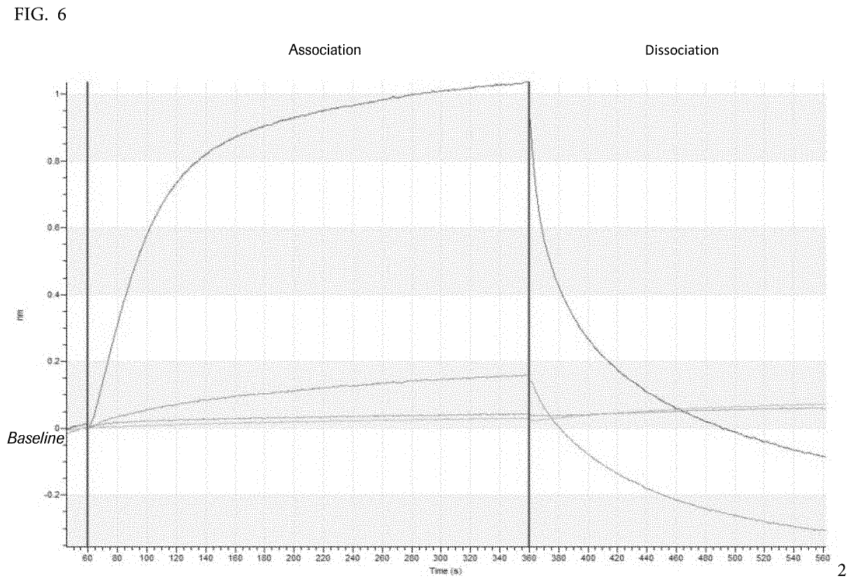

### Example 3: Bio-Layer Interferometry (BLI) Assay Using a Biosensor Seeded with Cells According to the Method of the Present Invention (See FIG. 6 for Results)

Pre-treatment of biosensor coated with cells to eliminate other cell activities (such as internalization) rather than binding. In order to study the cell surface molecular interaction with a ligand, the following 3 steps were added to the cell based BLI assay protocol:
1. Cold shock: Place the biosensors (surface down) into ice cold buffer or cell culture medium for about 5 mins before assay.
2. NaN₃ treatment: Place the biosensors (surface down) into buffer or cell culture medium contained 1-3 ng/mL NaN₃ for about 20 mins before assay.
3. Acetone treatment (cell fixing): Place the biosensors (surface down) into ice cold acetone for about 10 seconds before assay.

Bio-Layer Interferometry Assay:
Place the biosensors and reagent plate into the BLI instrument (follow BLI manual).
Define the assay steps and run the BLI.
General program: First dip Biosensors into the buffer wells→Baseline establishment.
Transfer Biosensors to sample wells (containing test compounds)→Association (binding observed)
Finally transfer Biosensors to buffer wells→Dissociation

### EMBODIMENTS

Embodiment I-1. A method for attaching cells to a biosensor surface (5) of a sensor (4) comprising:
a) providing a cell suspension in a liquid receiving unit (1), wherein the cell suspension forms a surface (7) to the exterior of the liquid receiving unit (1),
b) contacting the biosensor surface (5) with the surface (7) of the cell suspension in the liquid receiving unit (1) and
c) allowing the cells to settle on the sensor surface (5) by gravity, and allowing the cells to adhere to the biosensor surface (5).

Embodiment I-2. The method of Embodiment I-1, wherein the liquid receiving unit (1) keeps the cell suspension in a defined area/space through adhesion force and surface tension.

Embodiment I-3. The method of Embodiment I-1 or I-2, wherein the liquid receiving unit (1) comprises a structure selected from the group consisting of a capillary tube, a micro-groove, a micro-well, a micro-loop, a micro-wire spring, or a micro-protrude.

Embodiment I-4. The method of Embodiment I-3, wherein the liquid receiving unit (1) comprises a capillary tube (3) which is connected to a reservoir (2) to form a liquid receiving unit (1).

Embodiment I-5. The method of Embodiment I-3 or I-4, wherein the capillary tube (3) has an end opening (6) at which the cell suspension forms the surface (7) to the exterior of the liquid receiving unit (1) and said surface (7) is in contact with the sensor surface (5).

Embodiment I-6. The method of Embodiment I-4, wherein the capillary tube (3) has a hydrophobic zone at the end opening (6) to prevent the cell suspension from draining off.

Embodiment I-7. The method of Embodiments I-3-I-6, wherein the capillary tube (3) is at least filled with the cell suspension.

Embodiment I-8. The method of Embodiments I-1-I-7, wherein the liquid receiving unit (1) is arranged in array format comprising more than one liquid receiving unit (1), preferably the array format is a 96 unit plate format, more preferably a 96 unit IMAPlate™.

Embodiment I-9. The method of Embodiments I-1-I-8, wherein the sensor (4) is a needle-like sensor with a sensor surface (5).

Embodiment I-10. The method of Embodiments I-1-I-9, wherein the biosensor surface (5) is placed face-up.

Embodiment I-11. The method of Embodiments I-1-I-10, wherein in step c) the cells are allowed to settle for about 1-24 hours.

Embodiment I-12. The method of Embodiments I-1-I-11, wherein the biosensor surface (5) is coated with a biocompatible matrix to support cell attachment and cell growth.

Embodiment I-13. The method of Embodiments I-1-I12, wherein the biosensor surface (5) is coated with molecules which specifically interact with surface molecules of the cells to be immobilized on the sensor surface (5).

Embodiment I-14. A method for measuring molecular interactions between a test molecule and cells comprising:
a) immobilizing cells on a biosensor surface (5) according to the method of Embodiments I-1-I-13,
b) incubating the sensor (4) with the test molecule and
c) measuring interaction of the test molecule with the immobilized cells by appropriate methods.

Embodiment I-15. The method of Embodiment I-14, wherein the molecule is a bio-molecule.

Embodiment I-16. The method of Embodiment I-14, wherein the appropriate method is Bio-layer interferometry.

Embodiment I-17. The method of Embodiments I-14-I-16, wherein the test molecules are in reaction chambers of a multi well plate, preferably a 96 multi well plate.

Embodiment I-18. A kit for attaching cells to a biosensor surface (5) of a sensor (4) comprising a multi-unit plate comprising a plurality of liquid receiving units (1), wherein the liquid receiving units (1) have a reservoir part (2) and a capillary part (3) with an end opening (6), a set of biosensors (4) and a protocol for a method to attach cells to the biosensor surface (5) according to Embodiments I-1-I-13.

Embodiment I-19. The kit of Embodiment I-18, wherein the multi-unit plate is a IMAPlate™ and the sensor is a Bio-layer interferometry biosensor.

Embodiment I-20. The kit of Embodiment I-18 or I-19, further comprising a multi-unit plate which accommodates the set of biosensors (4) and spacers to connect the two multi-unit plates.

Embodiment I-21. The method of Embodiment I-12, wherein the biosensor surface (5) is pretreated with aceton before coated with biocompatible matrix.

1. A method for attaching cells to a biosensor surface (5) of a biosensor (4) comprising:
providing a cell suspension in a liquid receiving unit (1), wherein the cell suspension forms a surface (7) to the exterior of the liquid receiving unit (1);
contacting the biosensor surface (5) with the surface (7) of the cell suspension in the liquid receiving unit (1); and
allowing the cells to settle on the biosensor surface (5) by gravity, and allowing the cells to adhere to the biosensor surface (5).

2. The method of claim 1, wherein the liquid receiving unit (1) keeps the cell suspension in a defined area/space through adhesion force and surface tension.

3. The method of claim 1, wherein the liquid receiving unit (1) comprises a structure selected from the group consisting of a capillary tube, a micro-groove, a micro-well, a micro-loop, a micro-wire spring, or a micro-protrude.

4. The method of claim 1, wherein the liquid receiving unit (1) comprises a capillary tube (3) which is connected to a reservoir (2) to form a liquid receiving unit (1).

5. The method of claim 4, wherein the capillary tube (3) has an end opening (6) at which the cell suspension forms the surface (7) to the exterior of the liquid receiving unit (1) and said surface (7) is in contact with the sensor surface (5).

6. The method of claim 4, wherein the capillary tube (3) has a hydrophobic zone at the end opening (6) to prevent the cell suspension from draining off.

7. The method of claim 4, wherein the capillary tube (3) is at least filled with the cell suspension.

8. The method of claim 1, wherein the liquid receiving unit (1) is arranged in array format comprising more than one liquid receiving unit (1), preferably the array format is a 96 unit plate format, more preferably a 96 unit IMAPlate™.

9. The method of claim 1, wherein the sensor (4) is a needle-like sensor with a sensor surface (5).

10. The method of claim 1, wherein the biosensor surface (5) is placed face-up.

11. The method of claim 1, wherein the cells are allowed to settle for about 1-24 hours.

12. The method of claim 1, wherein the biosensor surface (5) is coated with a biocompatible matrix to support cell attachment and cell growth.

13. The method of claim 1, wherein the biosensor surface (5) is coated with molecules which specifically interact with surface molecules of the cells to be immobilized on the sensor surface (5).

14. A method for measuring molecular interactions between a test molecule and cells comprising:
immobilizing cells on a biosensor surface (5) according to the method of claim 1;
incubating the biosensor (4) with the test molecule; and
measuring interaction of the test molecule with the immobilized cells by an appropriate method.

15. The method of claim 14, wherein the appropriate method is Bio-layer interferometry.

16. The method of claim 14, wherein the test molecules are in reaction chambers of a multi well plate, preferably a 96 multi well plate.

17. A kit for attaching cells to a biosensor surface (5) of a biosensor (4) comprising:
a multi-unit plate having a plurality of liquid receiving units (1), wherein the liquid receiving units (1) have a reservoir part (2) and a capillary part (3) with an end opening (6) for receiving a cell suspension;
a set of biosensors (4); and
a protocol for a method to attach cells to the biosensor surface (5).

18. The kit of claim 17, wherein the multi-unit plate is an IMAPlate™ and the biosensor is a Bio-layer interferometry biosensor.

19. The kit of claim 17, further comprising a biosensor holder which accommodates the set of biosensors (4) and spacers to connect the biosensor holder and the multi-unit plate.

20. The method of claim 12, wherein the biosensor surface (5) is pretreated with acetone before being coated with the biocompatible matrix.
